(19) United States
(12) Patent Application Publication
(10) Pub. No.: US 2025/0222194 A1
(43) Pub. Date: Jul. 10, 2025

(54) SYSTEM AND METHODS AND PORTABLE BASE FOR BOWEL IRRIGATION

(71) Applicant: Coloplast A/S, Humlebaek (DK)

(72) Inventors: Niels HVID, Vedbaek (DK); Benjamin Holm Salka, Copenhagen N (DK); Nina Hoegh Jensen, Copenhagen N (DK); Stephanie Knoedler, Nivaa (DK)

(21) Appl. No.: 18/853,437

(22) PCT Filed: Mar. 29, 2023
(86) PCT No.: PCT/DK2023/050061

§ 371 Date: Oct. 2, 2024

(30) Foreign Application Priority Data
Apr. 8, 2022 (DK) PA202270196

(51) Int. Cl. A61M 3/02

(52) U.S. Cl. CPC A61M 3/0245; A61M 3/0237; A61M 2210/1064

(57) ABSTRACT

A portable base for a system for irrigation of the bowels of a user is provided. The portable base comprises a canister unit for an irrigation liquid, and a lid unit for engaging with the canister unit. In particular, the lid unit of the portable base comprises a rim portion comprising a rotatable skirt that is rotatably arranged around the rim portion. The portable base is adapted to be shifted between an unsealed configuration and a sealed configuration by rotating the skirt. The arrangement of the rotatable skirt on the rim portion facilitates easier shifting between the unsealed and sealed configuration, and vice versa, because less torque is required to be applied by the user for the sealing/unsealing. A system for irrigating the bowels of a user and a method of sealing the system and a method of irrigating the bowels of a user is also provided.

A portable base for a system for irrigation of the bowels of a user as well as a system and a method of bowel irrigation is provided. The portable base comprises a canister unit for an irrigation liquid, and a lid unit for engaging with the canister unit. In particular, the lid unit of the portable base has a rim portion with a rotatable skirt that is rotatably arranged around the rim portion. The portable base is adapted to be shifted between an unsealed (open) configuration and a sealed (closed) configuration (and vice versa) by rotating the skirt. The arrangement of the rotatable skirt on the rim portion facilitates easier shifting between the unsealed and sealed configuration, and vice versa, because less torque is required to be applied by the user for the sealing/unsealing of the portable base.

### BACKGROUND

Control of voluntary bowel functions is frequently limited or absent in patients suffering from certain disabilities, such as spinal injuries, multiple sclerosis, or spina bifida. Such lack of control of voluntary bowel functions typically results in faecal incontinence or intractable constipation, as patients have significantly reduced ability to sense the presence of faeces in the colon terminal part and the rectum and to sense the evacuation stimulus.

It is known to accomplish bowel emptying by irrigation (i.e. flushing) of the bowels via the rectum, by an irrigating fluid, such as tap water or saline, which is provided through an intermittent catheter with a tip which is configured and sized for insertion into the rectum (the rectum being the terminal section of the alimentary canal; from the sigmoid flexure to the anus), where it remains in a fixed position and held by an inflatable element, which may be inflatable by air or by water with a pump.

Once the rectum or stoma has been flushed with the irrigating liquid, the inflatable element is removed, and the liquid and faeces can evacuate. Some known irrigation systems suffer from being bulky, complicated, and difficult to handle and to clean properly after use. Moreover, some users of irrigation systems have limited dexterity and/or finger strength due to medical conditions and therefore may have difficulty in handling the components of the irrigation system, including particularly the opening, and closing of the container in the process of cleaning the water container and/or in the process of filling (or emptying) irrigation liquid from the container.

Thus, users of irrigation systems and health care professionals alike would welcome improvements in components of bowel irrigation systems.

### SUMMARY

In one aspect, the present disclosure provides a portable base for a bowel irrigation system. In another aspect, the disclosure relates to a bowel irrigation system including a portable base as described herein. In yet another aspect, the disclosure relates to a method of irrigating the bowels of a user with an irrigation system including the portable base disclosed herein. In yet another aspect, the disclosure relates to a method of sealing a canister unit of a bowel irrigation system disclosed herein. The aspects are further defined by the appended claims.

### BRIEF DESCRIPTION OF THE DRAWINGS

The accompanying drawings are included to provide a further understanding of embodiments and are incorporated in and constitute a part of this specification. The figures illustrate embodiments and together with the description explain principles of embodiments. Other embodiments and many of the intended advantages of all embodiments will be readily appreciated as they become better understood by reference to the following detailed description.

Various exemplary embodiments and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the effects or advantages shown. Conversely, an effect or an advantage described in conjunction with a particular embodiment is not necessarily limited to that embodiment and may be practiced in other embodiments even if not so illustrated, or if not so explicitly described.

The figures include the following:

FIG. 1 is a perspective view illustrating one embodiment of a portable base for a system for irrigating the bowels of a user in a closed/sealed configuration.

FIG. 2 is a cross-sectional view of one embodiment of a portable base in an unsealed/open configuration.

FIG. 2B is an enlarged portion of the cross-sectional view of the embodiment of a portable base according to FIG. 2.

FIG. 3 is a cross-sectional view of one embodiment of a portable base in a sealed/closed configuration.

FIG. 3B is an enlarged portion of the cross-sectional view of the embodiment of a portable base according to FIG. 3.

FIG. 3C is an enlarged portion of the cross-sectional view of the embodiment of a portable base according to FIG. 3.

FIG. 4 is another perspective view illustrating one embodiment of a portable base for a system for irrigating the bowels of a user in an open/unsealed configuration.

FIG. 5 is a schematic, perspective view of a simplified version of a portable base according to one embodiment.

FIG. 6 is a schematic, perspective view illustrating one embodiment of a system for irrigating the bowels of a user including one embodiment of a portable base.

FIG. 7 is a diagram illustrating the steps of a method of irrigating the bowels of a user with a bowel irrigation system incorporating one embodiment of a portable base.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” and equivalent, is used with reference to the orientation of the Figure(s) being described. Because components of embodiments can be positioned in a number of different orientations, the directional terminology is used for purposes of illustration and is not necessarily limiting. It is to be understood that other embodiments may be utilized, and structural or logical changes may be made without departing from the scope of the present invention. The following detailed description, therefore, is not to be taken in a limiting sense, and the scope of the present invention is defined by the appended claims.

It is to be understood that individual features of the various exemplary embodiments described herein may be combined with each other, unless specifically noted otherwise.

In this disclosure, solid and liquid wastes from the bowels of a person may be referred to as both “output,” “waste(s),” and “fluids” interchangeably. A person using the irrigation system may be referred to as “patient” or “user”. However, in some contexts, “user” can also relate or refer to a health care professional (HCP), such as a doctor/physician, nurse, or other person.

In this disclosure, ‘axial direction’ should generally be considered as vertical seen in relation to a top and bottom of the portable base. The radial direction should be understood as transverse to the axial direction. In some sentences, the words “inner” and “outer” can be used. These qualifiers should generally be perceived with reference to the radial direction, such that a reference to an “outer” element means that the element is farther away from a centre portion of a component than an element referenced as “inner”. In addition, “innermost” should be interpreted as the portion of a component forming a centre of the component and/or being adjacent to the centre of the component. In analogy, “outermost” should be interpreted as a portion of a component forming an outer edge or outer contour of a component and/or being adjacent to that outer edge or outer contour. In further analogy this is also the case about the use of the words “uppermost” and “lowermost”.

The use of phrases such as (but not limited to) “substantially” and “approximately” as qualifiers of certain features or effects in this disclosure, is intended to mean that any deviations are within tolerances that would normally be expected by the skilled person in the relevant field.

The use of the word “generally” as a qualifier to certain features or effects in this disclosure is intended to mean—for a structural feature: that a majority or major portion of such feature exhibits the characteristic in question, and—for a functional feature or an effect: that most outcomes involving the characteristic provide the effect, but that exceptionally some outcomes may not provide the effect.

The phrase “adjacent” should be interpreted as being “close to and/or in contact with, or temporarily brought into contact with, but not attached to [something].”

In the present disclosure, “portable” is to be understood as being easy to carry or move, so that a “portable” element can be used in different places and can be easily and conveniently transported. Particularly, this should be understood is relation to some types of available irrigation systems for the bowels of a user including components which are large and bulky and intended for bedridden and/or hospitalized users only.

In a first aspect, the present disclosure relates to a portable base for a system for irrigating the bowels of a user, comprising:
a canister unit comprising a volume for an irrigation liquid and a neck portion circumscribing an annular opening around a central vertical axis of the portable base;
a lid unit configured for closing of the annular opening of the canister unit, the lid unit having an axial extent along the central vertical axis between a top and a bottom of the lid unit, and comprising a rim portion and a rotatable skirt, the skirt being rotatably attached around the rim portion;
wherein the rotatable skirt of the lid unit and the neck portion of the canister unit comprise complementary threads;
wherein a sealing is provided at the rim portion and configured to seal between a radially inward facing surface of the neck portion of the canister unit and the lid unit; and
wherein the portable base is adapted to be shifted between an unsealed configuration and a sealed configuration by rotation of the rotatable skirt and engagement of the complementary threads of the rotatable skirt and the neck portion with each other, and vice versa.

The canister unit includes a volume for an irrigation liquid. The irrigation liquid can be temperate tap water or in some cases a saline physiological solution. The canister unit comprising the volume for the irrigation liquid can be considered a first major or primary component of the portable base. The canister unit includes a neck portion circumscribing an annular opening around a central vertical axis of the portable base. In embodiments, the neck portion comprises a vertical flange extending generally parallel to a central vertical axis of the portable base. In embodiments, an upper part (area or zone) of the flange includes a tapering, i.e., is at an angle to the central vertical axis. In embodiments, the neck portion transitions into a shoulder portion of the canister unit. The transition between the neck portion and the shoulder portion is located at a lower part (area or zone) of the neck portion. In embodiments, the shoulder portion of the canister unit (further or in turn) transitions into a sidewall of the canister unit. The transition between the shoulder portion and the sidewall of the canister unit is located at a lower part (area or zone) of the shoulder portion and can further be located radially outermost on the shoulder portion. In embodiments, the neck portion is offset radially inwardly from the sidewall of the canister unit by (or at) the shoulder portion and the neck portion is radially closer to the central vertical axis than is the sidewall of the canister unit. In embodiments, the neck portion includes one or more threads or thread segments in the form of protrusions and/or ledges extending generally radially from and perpendicular to the vertical flange.

In embodiments, the canister unit is adapted to stand by itself on a generally level surface of the surroundings. This is useful for making the portable base in its entirety stable when standing on a surface during use.

In embodiments, the sidewall of the canister unit extends substantially parallel to the central vertical axis. Thus, the canister unit generally takes the shape of a cylinder when viewed in cross-section.

In embodiments, the canister unit comprises a non-planar underside (or bottom).

The neck portion of the canister unit generally circumscribes the annular opening. The neck portion can be understood as delimiting, or defining or surrounding, the extent (or size) of the annular opening of the canister unit. Particularly, the annular opening provides a passage which is highly useful when cleaning or disinfecting the canister unit. In embodiments, the annular opening is relatively large, which greatly assists a user in cleaning the inside volume of the canister unit by inserting a hand or the fingers thereinto for cleaning. The annular opening can also at least partly provide a passage for filling and emptying irrigation liquid into and from the canister unit.

The portable base includes a lid unit configured for closing of the annular opening of the canister unit. The lid unit has an axial extent in a direction along the central vertical axis between a top and a bottom of the lid unit. Thus, the lid unit has a height in a direction parallel to the central vertical axis. The lid unit is provided with an external top surface (‘external’ to be understood as facing towards the surroundings away from the canister unit) and an internal bottom surface (‘internal’ to be understood as facing the volume for the irrigation liquid inside the canister unit). The use of the words ‘external’ and ‘internal’ shall also be understood in relation to the lid unit's being configured for attachment to (or engagement with) the canister unit over the annular opening (to close the canister unit).

The lid unit is provided with a rim portion. In embodiments, the rim portion is formed as a portion of a lateral wall of the lid unit. In embodiments, the rim portion is provided at a lower portion of the lateral wall (seen in relation to position on the central vertical axis). In embodiments, the lateral wall, or at least the rim portion thereof, is configured to be parallel to the central vertical axis. In embodiments, the rim portion of the lid unit is offset radially inward in relation to an upper portion of the lateral wall of the lid unit when viewed in relation to the central vertical axis. In embodiments, the lateral wall of the lid unit further defines or includes a shoulder at a transition between the rim portion and the upper portion. In embodiments, the lateral wall forms an annular sidewall of the lid unit.

The lid unit further includes a rotatable skirt. The rotatable skirt is rotatably attached around the rim portion of the lid unit. In embodiments, the rotatable skirt is arranged to be freely rotatable in both clockwise and counter-clockwise direction when (at least ‘as long as’) the lid unit is not engaged with (or placed on top of) the canister unit. The rotatable skirt is engaged with the rim portion in such a manner as to allow rotation of the skirt around the rim portion. In other embodiments, the rotatable skirt is engaged with the rim portion such as to allow a partial rotation around the rim portion (i.e., the skirt is configured to be rotated only a portion of a full circle around the rim portion).

In embodiments, the rotatable skirt includes a first flange which is configured to engage with a ledge of the rim portion of the lid unit to allow the first flange of the skirt to slide on the ledge during rotation of the skirt. In embodiments, the first flange extends in a direction parallel to a plane which is perpendicular to the central vertical axis. In embodiments, the first flange of the rotatable skirt extends radially inward towards the central vertical axis. In embodiments, the first flange extends radially inward towards the central vertical axis and is latched (held in place) between the ledge of the rim portion and a shoulder of the upper portion of the lateral wall of the lid unit. In embodiments, the shoulder is located at a transition between an upper portion of the lateral wall of the lid unit and the rim portion of the lid unit. In embodiments, the transition is defined at a location where the rim portion is radially inwardly offset in relation to the upper portion. Thus, in embodiments, the shoulder provides a downwardly facing surface of the upper portion of the lateral wall of the lid unit. In embodiments, the shoulder forms the transition between the upper portion of the lateral wall of the lid unit and the rim portion of the lid unit. In embodiments, the shoulder is provided as an annular shoulder extending annularly entirely along the upper portion (full circle of the lid unit). In embodiments, a radial extent of the shoulder in a direction perpendicular to the central vertical axis can be configured within a range of 5-30 mm (along the downwardly facing surface of the upper portion).

In embodiments, the rotatable skirt includes a second flange. In embodiments, the second flange extends in a direction parallel to a plane which is generally perpendicular to the central vertical axis. In embodiments, the second flange of the rotatable skirt extends radially inward towards the central vertical axis. In embodiments, the second flange is configured to engage with and slide in the neck portion of the canister unit during closing (sealing) and opening (unsealing) action. In embodiments, the second flange is configured to engage with and slide in a groove or track of the canister unit. In embodiments, the groove or track is provided in the neck portion of the canister unit. In embodiments, the groove or track is formed between (or by) the neck portion and the shoulder portion of the canister unit.

In embodiments, the groove or track of the canister unit is delimited and/or defined by at least a portion of a first thread provided on the neck portion of the canister unit and the shoulder portion of the canister unit. In embodiments, the second flange of the rotatable skirt forms at least a portion of a second thread of the rotatable skirt. In embodiments, the second flange of the rotatable skirt forms the second thread of the rotatable skirt. The first thread of the canister unit and the second thread of the rotatable skirt are configured as complementary threads. Thereby, the first and second threads are suitable for and configured to go into engagement with each other to attach the lid unit to the canister unit.

In embodiments, the second flange of the rotatable skirt has a shorter radial extent than the first flange of the rotatable skirt. In embodiments, the first flange can be understood to help provide each of an attaching function, a movement controlling function, and a sliding function, or capability, of the rotatable skirt in relation to the rim portion of the lid unit (the first flange being engaged and held “in place” by the ledge and the shoulder). In embodiments, the engagement between the first flange of the rotatable skirt, the ledge in the rim portion, and the shoulder, allows the rotatable skirt to rotate around the central vertical axis and/or prevents the rotatable skirt from disengaging from the lid unit. In embodiments, a notch (e.g., U- or V-shaped) is provided in the rotatable skirt. In embodiments, the notch is provided or spatially defined by the first flange, the second flange, and a lateral wall of the rotatable skirt. In embodiments, the notch forms at least a part of a second thread of the rotatable skirt. In one embodiment, the notch of the rotatable skirt forms the second thread of the rotatable skirt.

A sealing is provided at the rim portion and configured to seal between a radially inward facing surface of the neck portion of the canister unit and the lid unit. In embodiments, the sealing is attached to the rim portion of the lid unit and brought into abutment with the neck portion of the canister unit when the lid unit is placed on the neck portion. A seal (sealing effect) is effectuated when the sealing is urged or pressed into engagement with the neck portion by rotation of the rotatable skirt to cause the complementary threads to come into engagement with each other and shift the portable base to the sealed configuration. During the sealing (closing) action, the engagement of the complementary threads with each other, and the force incurred on the components when the user rotates the rotatable skirt, results in an axially downward movement of the lid unit in relation to the canister unit, which in turn causes the sealing to be pressed into leak-tight engagement with the neck portion and creates a seal (sealing effect) between the lid unit and the canister unit. The rotation of the skirt and the resulting axially downward movement of the lid unit leads to pressure being applied on the material of the sealing. One advantageous effect of the provision of the sealing is that the irrigation liquid in the canister unit of the portable base can be pressurized, and maintained pressurized, independently of tubing of a system for irrigation the bowels of a user.

By rotation of the rotatable skirt the first thread of the canister unit and the second thread of the rotatable skirt, being configured as complementary threads, are adapted to come into engagement with each other, which both results in the securing of the lid unit onto the canister unit and shifting of the portable base to the sealed (closed) configuration. This is because the combination of the rotational movement of the skirt and the engagement of the complementary first and second threads causes an axially downward movement of the lid unit which in turn causes the sealing to seal between the lid unit and the canister unit. In embodiments, the first thread of the canister unit is adapted to provide a “male” part of the complementary threads. In embodiments, the second thread of the rotatable skirt is adapted to provide a “female” part of the complementary threads (e.g., in the form of a notch, as described above). In embodiments, the rotatable skirt includes at least one second thread provided on (or in) a radially inward facing surface of the skirt. In embodiments, exactly one second thread is provided. The one second thread can be configured to extend around a portion of an inward facing surface of the rotatable skirt. In embodiments, the second thread of the rotatable skirt is provided wholly or partially by the second flange of the rotatable skirt described above. Other implementations of complementary threads are acceptable.

In embodiments, the first thread of the canister unit includes three or more evenly distributed, individual, and finite thread segments. In embodiments, at least the first thread segment(s) on the canister unit are sloped or tapered such that by the rotational movement of the skirt comprising the second thread(s) in relation to the complementary first, sloping thread segment(s) on the canister unit, the lid unit is moved in an axial direction parallel to the central vertical axis (opening or closing action). In embodiments, the first thread(s) on the neck portion comprise at least three spaced apart thread segments provided on a radially outward facing surface of the neck portion. In embodiments, the second thread segment(s) on the rotatable skirt are sloped or tapered.

In embodiments, the rotatable skirt includes a gripping tab extending in a direction radially outward in relation to the central vertical axis. In embodiments, the gripping tab advantageously extends radially beyond the annular sidewall of the lid unit and radially beyond the shoulder portion of the canister unit. A portion of the gripping tab extending radially beyond the annular sidewall and beyond the shoulder portion can be shaped to align with the sidewall of the canister unit. Thereby, access to and movement of the tab is made easy and unobstructed. In embodiments, the gripping tab on the rotatable skirt is configured to generally align with a second tab (or finger-hold) on the sidewall of the canister unit when the portable base is in the sealed configuration. Conversely, the gripping tab on the rotatable skirt and the tab or finger-hold on the canister unit can be configured to not align when the portable base is in the unsealed configuration.

A particular advantageous effect of embodiments of the above-described structure of the sealing of the portable base is that, for closing and opening of the canister unit with the lid unit, it is not necessary to overcome a frictional force required to rotate the sealing. The sealing itself does not have to be moved (rotated) annularly around the central vertical axis during the rotation of the rotatable skirt of the lid unit. Instead, in embodiments, the sealing only needs to move a relatively short distance in the vertical direction by the force incurred by the user by rotating the rotatable skirt of the lid unit. Thus, a portable base for a system for irrigating the bowels of a user is provided, wherein the force needed to seal and unseal (close and open) the container (canister unit) containing the volume of irrigation liquid is reduced, in that it is not necessary for the user to overcome a combination of both the frictional force for rotating the sealing itself and the normal force required to “lift” (or “push down”) the sealing in a vertical direction parallel to the central vertical axis. According to the disclosure, it is only necessary to overcome the normal force for moving the sealing a short distance in the vertical direction. This is particularly useful as it allows for easier unlatching or unfastening of the lid unit in its entirety. It also allows for the provision of an opening for an irrigation liquid container which can be of relatively large diameter or size thereby making it easier for the user to clean the inside of the container volume and/or potentially to fill the container with irrigation liquid for use. Making cleaning of the components of the portable base easier, particularly cleaning of the canister unit, holding the irrigation liquid, by allowing the user to get a hand inside the canister unit for the cleaning procedure, is useful and advantageous and provides a solution that is both convenient and intuitive for the user. Moreover, lowering or reducing the necessary force required to open and close the container is also advantageous and useful in that users of irrigation systems often lack hand dexterity due to various medical conditions and/or plainly lack sufficient physical strength. Additionally, the mere reason that some users have a relatively small hand size, can make it difficult to rotate closures or lids on containers having relatively large openings/lids. According to the present invention, the elimination of the need to overcome a frictional force required for rotation of a sealing in an irrigation liquid container, alleviates, or at least reduces, each of these issues and helps provide the option of providing a container having a large opening, which in turn helps provide for the easy cleaning solution.

It is thus to be understood that a portable base for a system for irrigating the bowels of a user is provided, in which closing and opening (and sealing) of the container for holding the irrigation liquid is more easily done by virtue of the rotatable skirt being rotatable on the lid unit in a direction around the central vertical axis. According to the disclosure, any need for simultaneously forcing the sealing to be rotated for closing and opening (“alongside” with the entire lid unit), can be avoided. The construction facilitates that only the axial ‘lifting up’ (opening) or ‘pushing down’ (closing) of the sealing takes place instead of having to additionally rotate the sealing for the opening/closing (and thus the sealing/unsealing) action to occur. In addition, the obtained axial ‘lifting up’ (opening action) of the sealing by rotating the rotatable skirt, allows for internal air-pressure in the portable base to be relieved (released or equalized) already prior to the lid unit being entirely freed from the engagement between the complementary threads of the rotatable skirt and the lid unit. The achieved result of ‘lifting up’ the sealing, and the “early” relief or “initial” equalization of pressure in the canister unit, at slightly different moments in time, also helps reduce or eliminate a frictional force required to perform any subsequent additional rotation/turning of the lid unit for its removal from the annular opening of the canister unit (i.e., “finally” disengaging the complementary threads from each other). It is to be understood, that the portable base shifts from the sealed to the unsealed configuration due to the rotation of skirt, the rotating movement causing a “lifting” or “pulling” up (upward movement) of the sealing, but not a rotation of the sealing. After the opening action, and relief of pressure in the canister unit, any force required for the subsequent removal of the lid unit from the canister unit is negligible, and thus much easier overcome by users. Additionally, the upward axial movement of the sealing can advantageously be assisted by adapting (or customizing) the size, shape, and/or form of the complementary threads, particularly the first thread(s) or thread segment(s) of the canister unit, for example, by way of tapering or sloping of this/these. In some embodiments, additional means for assisting the user in equalizing the pressure inside the canister unit and the surroundings can be incorporated.

It is an additional advantage of the present disclosure that no or only little rotation or turning of the entire lid unit is needed to unseal/open and/or seal/close the canister unit with the lid unit. Firstly, in analogy to above, avoiding rotation of the lid unit in its entirety means that less force is required to be applied by the user for the opening and/or closing of the container containing the irrigation liquid. Secondly, avoiding rotation of the lid unit is advantageous in embodiments of an irrigation system, wherein one or more components of the system are incorporated into the lid unit (for example control and power components and/or a pump unit) and further in embodiments wherein tubing for the irrigation liquid is attached to the portable base via (in engagement with) the lid unit. In such cases, the portable base including the lid unit and the canister unit makes handling of the different components of an irrigation system in relation to each other easier and less cumbersome. As an example, potential twisting or unintended winding of irrigation liquid tubing extending from the lid unit, caused by rotation of the lid during the opening and closing of the portable base can be avoided, thus reducing the potential risk of malfunction and/or of impeding of liquid flow in the tubing. Among other advantages, the lid unit can additionally house one or more delicate control components in one or more internal chambers, e.g., a CPU and/or other electronics, whereby it is useful to avoid movements of the entire lid unit to reduce unnecessary shifting and stress on the housed components from the externally incurred rotation forces. The option of attaching the lid unit on the canister unit and avoiding a substantial rotation of the entire lid unit itself, further adds flexibility for a user of an irrigation system incorporating the portable base in terms of easing requirements for putting together the components of the system in a specific, and/or, predetermined order. Thus, in embodiments according to the disclosure it does not matter if, for example, the irrigation liquid tubing has been inadvertently attached to the connection on the lid unit before closing/sealing of the lid unit onto the canister unit, because the lid unit itself with the attached tubing is not rotated during the closing/sealing action, only the rotatable skirt is rotated or turned to close and seal the portable base.

In embodiments, the portable base is configured to be pressurized in the sealed configuration. When the portable base is shifted to the sealed and closed configuration by rotating the rotatable skirt and engaging of the complementary threads of the canister unit, the sealing being in engagement with the neck portion of the canister unit provides an air- and/or liquid-tight seal between the lid unit and the canister unit. Thus, in the sealed configuration, the portable base can be pressurized by an air- (e.g., vacuum-) pump (e.g., incorporated in a system for irrigating the bowels of a user). The pressurization possibility facilitates setting irrigation liquid in the canister unit under a desired operating pressure suitable for irrigating the bowels of a user, by means of an air-pump rather than by using a water-pump for pumping the irrigation liquid out of the portable base and to the anal probe (by physically propelling or pushing the liquid). In embodiments, the portable base is configured for a liquid operating pressure of between 6.9-15.9 kPa (approximately 1.0-2.3 psi). In one embodiment, the operating pressure is configured at a maximum of approximately 13.8 kPa (˜2.0 psi). In embodiments, specific implementations of the first thread(s) or thread segment(s), and the second thread(s), of the complementary threads, can be adapted to help accommodate the desired air-pressurization capacity of the portable base (i.e., adapted to help operate the system with the desired irrigation liquid pressure). Again, such adaptation can be facilitated inter alia by varying the number of individual threads and/or segments, the tapering/sloping, size/dimensions, and the materials of the complementary threads and additionally and/or alternatively by customizing the sealing type and dimensions.

An advantage of the portable base according to the disclosure is that it allows for using an air-pump (e.g., vacuum-pump) to pressurize the irrigation liquid in the canister unit. Due to the way the sealing and the means for opening and closing (sealing/unsealing) combine to facilitate (de-)pressurization of the portable base, the invention allows for the use of an air-pump with a system for irrigating the bowels of a user as opposed to using a water (irrigation liquid) pump that physically propels the liquid. Thereby, the use of a water pump for pressurization of the irrigation liquid, and hence the disadvantages involved therewith, is avoided. In a water pump, the irrigation liquid is propelled through the water pump under pressure to irrigate the bowels. Propelling irrigation liquid/water through a water pump incurs a necessary requirement of frequent and thorough cleaning of the water-pump to prevent growth of microorganisms in the pump. With the portable base of the present disclosure, which allows the portable base to be pressurized while at the same time reducing the force needed to seal/unseal (close/open) the canister unit, a bowel irrigation system incorporating the disclosed portable base can rely on the use of an air pump and thus avoid sending irrigation liquid through a pump unit, in turn avoiding the need for frequent cleaning action. It is thus understood that the disclosure also provides a portable base for a system for irrigating the bowels of a user, in which no physical contact between the irrigation liquid and the pump unit is necessary. This advantageously prevents contamination of the pump unit and further offers a solution wherein no moving parts of the pump unit are subjected to contact with the irrigation liquid or to wear caused by propelling thereof.

In embodiments, the annular opening of the canister unit has a diameter of between 100-150 mm. In one embodiment, the annular opening of the canister unit has a diameter of 120 mm. These embodiments are particularly suitable for providing a possibility of thoroughly cleaning the insides of the canister unit, e.g., by allowing a user to insert a hand entirely through the annular opening or alternatively a brush or other cleaning tool.

In embodiments, the sealing is provided at a location of the rim portion being closer to the bottom of the lid unit than to the top of the lid unit. In embodiments, the sealing is configured to seal against a lowermost part of the radially inward facing surface of the neck portion of the canister unit in the sealed/closed configuration of the portable base. Different to other embodiments of the disclosure, these embodiments ensure that the sealing is moved a relatively long vertical distance while being in abutment with the neck portion of the canister unit during the closing and/or opening action and can therefore be adapted to require application of a relatively larger force for moving the lid unit in the vertical direction, should this be desired or needed. One reason would be to provide an option for adapting or customizing the principal force needed to seal between the lid unit and the canister unit, in turn e.g., allowing for pressurization of an irrigation system with a relatively higher air-pressure acting on the liquid in the canister unit.

The sealing is provided at the rim portion and configured to seal between a radially inward facing surface of the neck portion of the canister unit and the lid unit. In embodiments, the radially inward facing surface is parallel to the central vertical axis. In embodiments, the radially inward facing surface is an inner annular rim of the neck portion. In embodiments, the inner annular rim has an extent in a direction parallel to the central vertical axis of between 5 mm and 30 mm. In one specific implementation, the vertical extent of the inner annular rim is 10 mm. In another specific implementation, the vertical extent of the inner annular rim is 15 mm. In another specific implementation, the vertical extent of the inner annular rim is 20 mm. In yet another specific implementation, the vertical extent of the inner annular rim is 30 mm. Such implementations provide a good abutment surface for the sealing on the rim portion of the lid unit against the neck portion of the canister unit. In embodiments, an uppermost part of the radially inward facing surface of the neck portion of the canister unit is angularly offset seen relative to the central vertical axis, for example, such that the uppermost part tapers or slopes away from the central vertical axis. This implementation can be useful e.g., for assisting in guidance of the rim portion of the lid unit into correct position on top of the canister unit, in turn guiding the sealing into correct location.

In some embodiments, the sealing comprises an O-ring. In embodiments, the sealing is an O-ring. The O-ring is an annular sealing ring, e.g., made from a rubber-based or silicone-based material (or any other suitable polymeric material), and which extends around the rim portion of the lid unit. In embodiments, the O-ring is suitably made from a specific silicone material. The O-ring is suitably attached to the rim portion by an adhesive. Alternatively, the O-ring can be mechanically fastened or attached to the rim portion or by a combination of adhesive and mechanical fastening.

In embodiments, the lid unit includes a through-going passage for filling an irrigation liquid into the volume in the canister unit (i.e., through the lid unit). The through-going passage is provided generally parallel to the central vertical axis. In embodiments, access to the irrigation liquid passage is possible by removing a cap, which can be screwed or press-fitted onto the lid unit. In embodiments, the cap is provided in a top of the lid unit. In embodiments, the cap comprises an internal sealing (rubber or silicone gasket or gasket-like sealing) for providing an air- and/or liquid-tight seal with the lid unit and in turn the canister unit to assist in the pressurization of the portable base when it is to be used for irrigation. The cap facilitates filling (or in some cases emptying) of the canister unit with irrigation liquid without requiring removal or opening (unscrewing/unsealing) of the lid unit entirely from the canister unit. Also, the internal sealing of the cap can facilitate (de-)pressurization of the portable base independently of removal of the lid unit from the canister unit.

In embodiments, the lid unit includes at least one internal chamber. The at least one internal chamber is adapted to contain one or more control elements of a system for irrigating the bowels of a user. In embodiments, one or more control elements are held in the internal chamber and/or partially on an outer surface of the lid unit. In embodiments, a top of the lid unit comprises one or more connections and/or interfaces (e.g., spigots or studs) for engagement and correspondence with other elements and/or components of a system for irrigation the bowels of a user. In embodiments, the lid unit includes a passage for allowing irrigation liquid from the canister unit to exit from the canister, typically by means of a pumping action provided by a pump of the system for irrigating the bowels of a user. In one embodiment, the lid unit includes two separate, internal chambers. In embodiments, the lid unit includes more than two separate, internal chambers. In embodiments, one internal chamber of the lid unit includes or houses a pump and forms part of a system for irrigating the bowels of a user.

In embodiments, the components of the portable base are made from materials suitable for handling tap water with a temperature of between 5 and 60 degrees centigrade, such as between 30 and 45 degrees centigrade. In embodiments, the canister unit is made from materials suitable for handling temperatures up to approximately 100 degrees Celsius for cleaning purposes. In embodiments, the components of the portable base should be configured to support a system for irrigating the bowels of a user which allows a flow within the levels of approximately 1000 mL/min at 0 bar (0 kPa) and 250 mL/min at 0.1 bar (10 kPa).

The portable base of the disclosure offers the user increased flexibility and increased convenience, particularly, but not exclusively, when used in a system for irrigating the bowels of a user. Advantages include reduction of complexity of the portable base and minimization of requirements or increased ease of cleaning, drying and other maintenance thereof. Further advantageously, the portable base of the present disclosure can be seen to provide a solution without any electrical connections/engagements between the canister unit and the lid unit, thereby minimizing risks of faulty electrical connections and/or other electrical malfunctions.

In another aspect, the disclosure relates to a bowel irrigation system including the portable base described herein and further includes the following components: irrigation tubing comprising a first end and a second end, the first end of the irrigation tubing configured to be connected to the lid unit of the portable base, at least one anal probe configured to be connected to the second end of the irrigation tubing, optionally, a remote control for controlling one or more control elements of the system, optionally, a set of instructions for use, and optionally, a container for storing the components of the system (between uses). In embodiments, the lid unit of the portable base includes one or more internal chambers as described herein, wherein at least a pump unit and control components or an element adapted to control the irrigation procedure is provided in one or more of the internal chambers. An electrical supply unit for the irrigation system can also be incorporated or can be provided separately from the system itself.

In yet another aspect, the disclosure relates to a method of irrigating the bowels of a user with a bowel irrigation system including the portable base disclosed herein, comprising the steps of:
(a) providing the canister unit and the lid unit of the portable base;
(b) providing additional components of a bowel irrigation system;
(c) providing a volume of irrigation liquid in the canister unit;
(d) placing the lid unit on the neck portion of the canister unit to close the annular opening of the canister unit;
(e) rotating (cause to turn about the central vertical axis, at least partially) the rotatable skirt on the rim portion of the lid unit such that complementary threads of the rotatable skirt and the canister unit engage with each other to shift the portable base to a sealed configuration; and
(f) irrigating the bowels of the user.

In yet another aspect, the disclosure relates to a method of sealing a canister unit of a bowel irrigation system including the portable base disclosed herein, comprising the steps of:
(a) providing the canister unit and the lid unit of the portable base;
(b) providing additional components of a bowel irrigation system;
(c) providing a volume of irrigation liquid in the canister unit;
(d) placing the lid unit on the neck portion of the canister unit to close the annular opening of the canister unit; and
(e) rotating the rotatable skirt on the rim portion of the lid unit such that complementary threads of the rotatable skirt and the canister unit engage with each other to shift the portable base to a sealed configuration.

The method according to this aspect is advantageous in that using a portable base as disclosed above provides the effects and benefits to the user described in detail for the portable base device, including (but not limited to) ease of sealing and opening the canister unit using significantly reduced physical force, and further providing significantly easier access for cleaning of the canister unit.

### DETAILED DESCRIPTION OF THE DRAWINGS

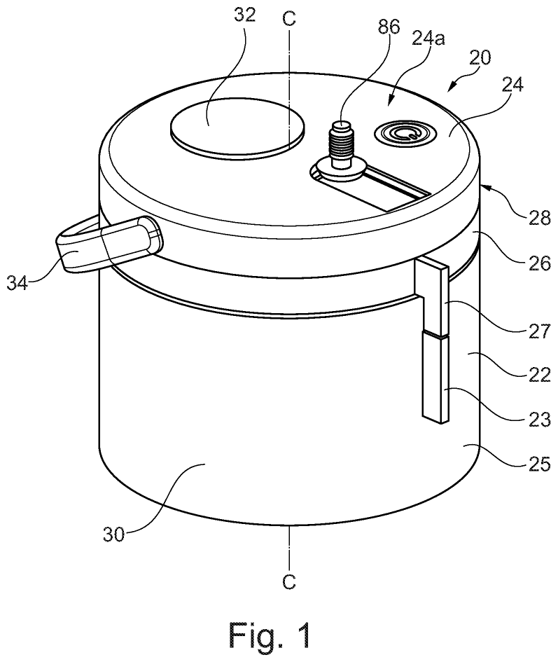

FIG. 1 is concerned with a first aspect of the present invention and is a perspective view illustrating one embodiment of a portable base 20 for a system for irrigating the bowels of a user. The portable base includes a canister unit 22, which is self-supporting and a lid unit 24. In the perspective view of FIG. 1, the portable base 20 is shown in a sealed (or closed) configuration with the lid unit 24 is placed “on top of” and is closing the canister unit 22. The canister unit 22 is adapted to hold a volume of irrigation liquid (not shown) inside the canister. It is to be understood that the lid unit 24 is removably connected to the canister unit 22. The lid unit 24 includes a rotatable skirt 26. The lid unit 24 is adapted to be released and subsequently removed from the canister unit 22 by rotation of the rotatable skirt 26. The rotatable skirt 26 can be at least partly rotated without rotating other portions or parts of the lid unit 24 and the canister unit 22. A gripping tab 27 extends out from the rotatable skirt 26 and is illustrated to substantially align with a finger-hold 23 of the canister unit 22. Particularly, the gripping tab 27 and the finger-hold 23 of the canister unit are configured to align with each other in the sealed/closed configuration of the portable base 20. By moving the gripping tab 27 of skirt 26 in relation to the finger-hold 23 of the canister unit 22 the rotatable skirt 26 can be moved in a direction around the central vertical axis and thereby be released from engagement with the canister unit 22 (complementary threads provided on the rotatable skirt 26 and on the neck portion of the canister unit 22 thereby release from each other). After such rotational action of the rotatable skirt 26, and the release of threads of the rotatable skirt from threads of the canister unit 22, the lid unit 24 can be removed from the canister unit 22 by lifting it off therefrom. In the embodiment shown in FIG. 1, the lid unit 22 includes a handle 34 attached to the lid unit which can be used when lifting off the lid unit 24 from the canister unit 22 and for moving of the portable base around when it is in the sealed configuration.

The lid unit 24 can be configured to hold one or more control elements or control components of a bowel irrigation system. The lid unit 24 includes a lateral wall. In the illustrated embodiment, the lateral wall forms an annular sidewall 28 of the lid unit 24. In the illustrated embodiment, the finger-hold 23 extends laterally out from a sidewall 30 of the of the canister unit 22. Further, in the embodiment of FIG. 1, the lid unit 24 is shown including a cap 32 which can be opened to supply an irrigation liquid through an opening in the lid unit 24 (opening not visible in FIG. 1 as it is covered by the cap 32) and into the volume of the canister unit 22. The cap 32 is provided in a top 24a of the lid unit 24 and is removable therefrom, e.g., by unscrewing the cap. In FIG. 1, the sidewall 30 of the canister unit 22 is substantially straight and parallel to a central vertical axis C-C of the portable base 20.

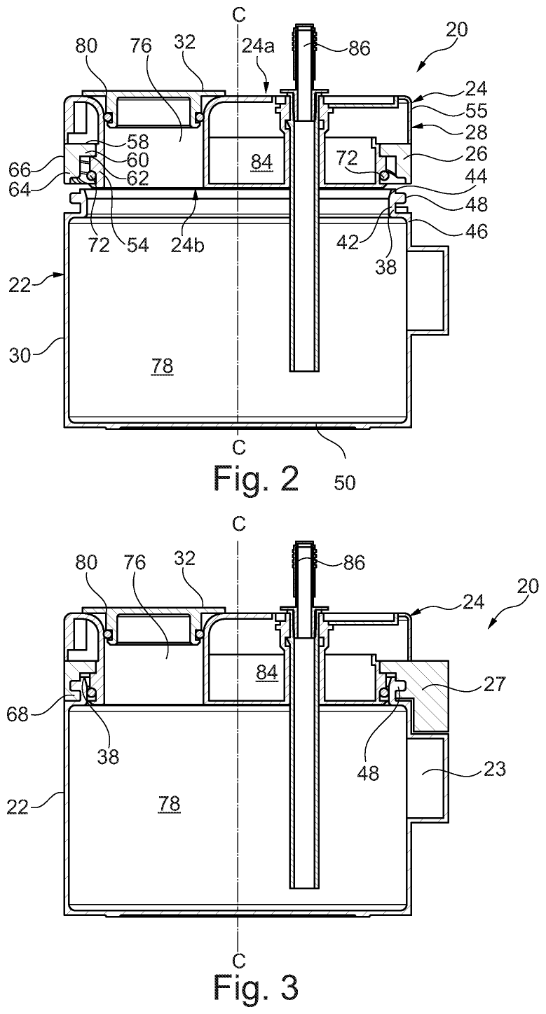

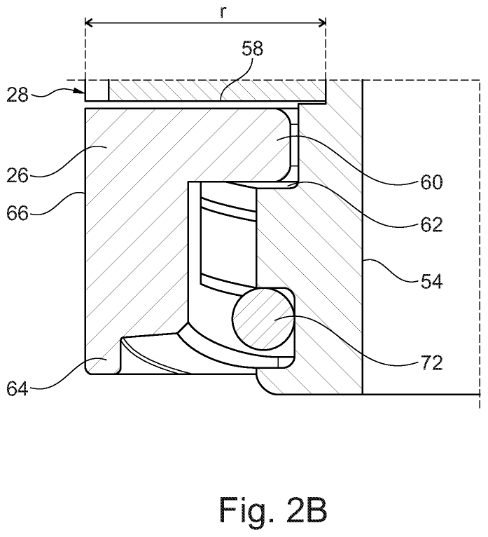

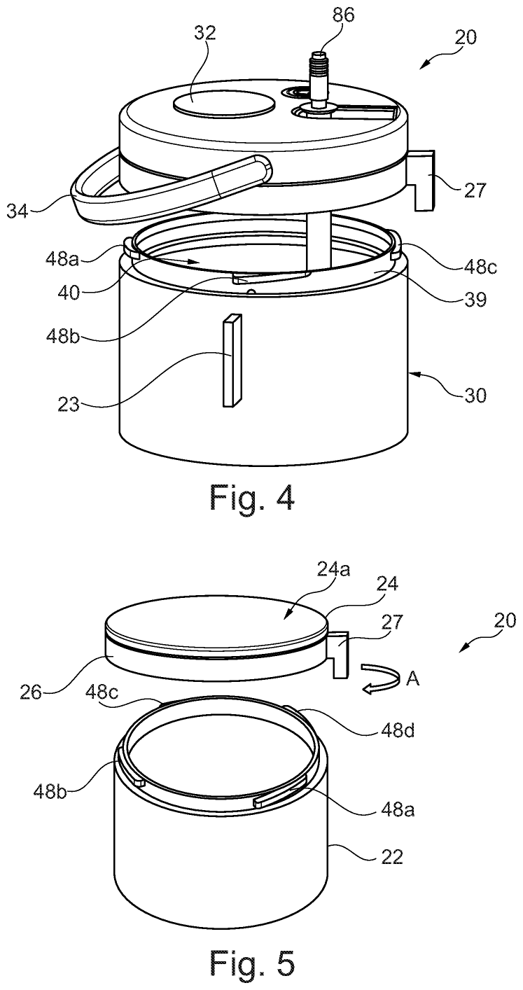

FIG. 2 is a cross-sectional view of one embodiment of the portable base 20 in an unsealed/open configuration, i.e., wherein the lid unit 24 is not in engagement with the canister unit 22. The canister unit 22 includes a neck portion 38 which in the illustrated view circumscribes an annular opening 40 (FIG. 4) around the central vertical axis C-C of the portable base 20. The neck portion 38 includes a vertical flange 42 extending parallel to the central vertical axis C-C. In FIG. 2, an upper part or end of the vertical flange 42 includes a tapering 44. The neck portion 38 transitions into a shoulder portion 46 of the canister unit 22. The transition between the neck portion 38 and the shoulder portion 46 is located at a lower part of the neck portion 38. The shoulder portion 46 of the canister unit 22 transitions into sidewall 30 of the canister unit 22. In the embodiment of FIG. 2, the neck portion 38 is offset (or extends) radially inwardly from the sidewall 30 of the canister unit 22 at the shoulder portion 46 and is therefore located radially closer to the central vertical axis C-C than is the sidewall 30 of the canister unit 22. The neck portion 38 includes a protrusion 48 extending radially outward from and perpendicular to the flange portion 42. The protrusion 48 provides at least a portion of a first thread or thread segment of the neck portion 38 of the canister unit 22. In embodiments, the protrusion or first thread 48 is extending from vertical flange 42 of the neck portion 38 annularly around (an entirety) of the annular opening 40 of the canister unit 22. The neck portion 38 of the canister unit 22 generally circumscribes the annular opening 40. The neck portion can be seen to delimit the extent of (and thus a size) the annular opening 40 of the canister unit 22.

In FIG. 2, the canister unit 22 comprises a non-planar underside 50. The non-planar underside 50 includes an apex 52 that coincides with the central vertical axis C-C, the apex 52 being closer to the top portion of the canister unit 22 than any other location on the underside 50 of the canister unit 22. It is to be understood that the non-planar underside 50 forms a dome shape in a three-dimensional perspective, i.e., the apex 52 forms the apex of the underside in any direction, e.g., perpendicular to the direction of the “paper”.

In FIG. 2, the lid unit 24 is shown “on top” of the canister unit 22 and ready for closing the annular opening 40. The lid unit 24 has an axial extent along the central vertical axis C-C between a top 24a and a bottom 24b of the lid unit 24. The lid unit 24 includes a rim portion 54. The lid unit 24 further includes the rotatable skirt 26 (see also FIG. 1). The rotatable skirt 26 is rotatably attached to the lid unit 24 around the rim portion 54. The rim portion 54 is part of the lateral wall 28 of the lid unit 24. In FIG. 2, the rim portion 54 is provided at a lower portion of the lateral wall 28 (seen in relation to positions on the central vertical axis C-C). In the illustrated embodiment, the rim portion 54 is parallel to the central vertical axis C-C and is offset radially inward in relation to an upper portion 55 of the lateral wall 28 (seen in relation to the central vertical axis C-C). In the embodiment of FIG. 2, the lateral wall 28 of the lid unit 24 also includes a shoulder 58 at a transition between the rim portion 54 and the upper portion 55 of the lateral wall 28.

The rotatable skirt 26 is rotatably attached around the rim portion 54 of the lid unit 24. The rotatable skirt is at least partially rotatable in both clockwise and counter-clockwise direction when the lid unit 24 is not engaged with (on top of) the canister unit 22. The rotatable skirt 26 is configured to engage with the rim portion 54 while allowing at least partial rotation of the skirt 26 around the rim portion 54.

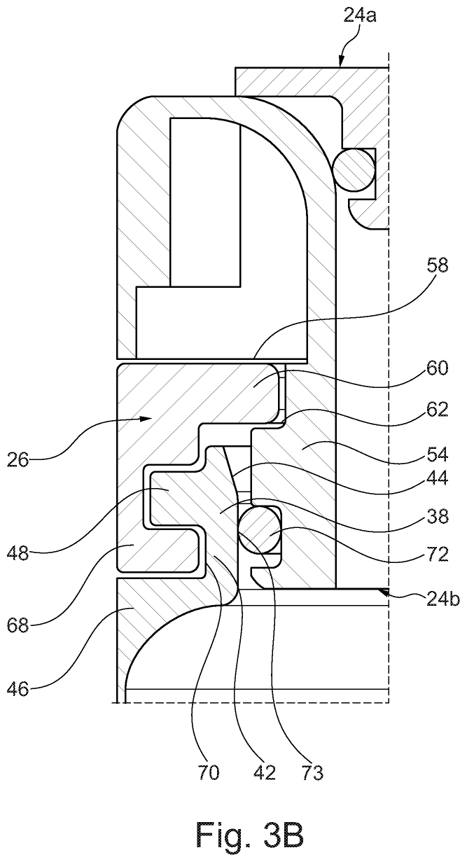

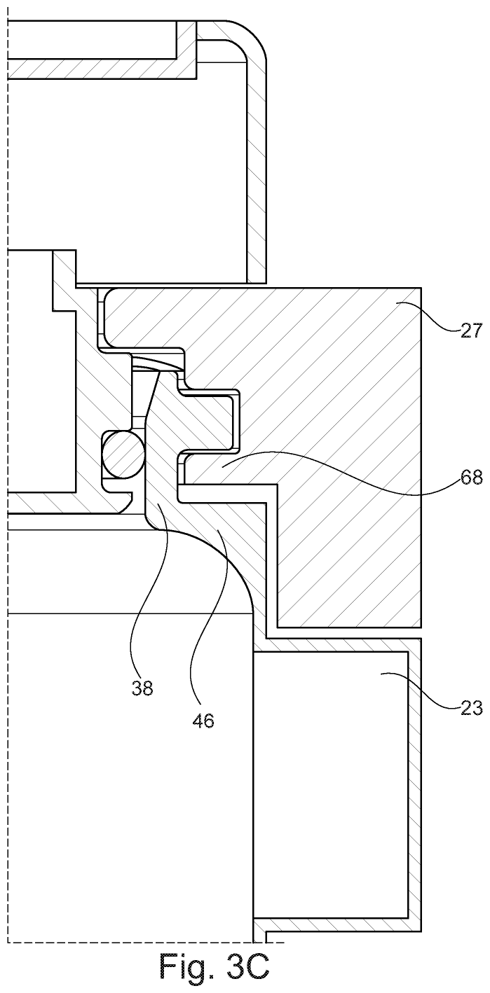

In the embodiment of the enlarged sectional view of FIG. 2B, the rotatable skirt 26 includes a first flange 60 which engages with a ledge 62 of the rim portion 54 to allow the first flange 60 to slide on (along) the ledge 62 during rotation of the skirt 26. The first flange 60 extends in a direction parallel to a plane which is perpendicular to and extends radially inward towards the central vertical axis C-C. In the embodiment of FIG. 2B, the first flange 60 is additionally latched between the ledge 62 in the rim portion 54 and the shoulder 58 of the upper portion of the lateral wall 28 of the lid unit 24. The shoulder 58 is located at the transition between the upper portion 55 and the rim portion 54 of the lateral wall 28. The transition is defined at a location where the rim portion 54 is radially inwardly offset towards the central vertical axis C-C in relation to the upper portion of the lateral wall 28 (FIG. 2). The shoulder 58 can be seen to have a radial extent “r” in a direction perpendicular to the central vertical axis C-C. In the cross-sectional views of FIGS. 2 and 2B, a portion 64 of second flange or second thread 68 (FIG. 3) of the rotatable skirt 26 can be seen extending downwardly at an outer periphery 66 of the rotatable skirt 26. In the embodiment of FIG. 2B, the first flange 60 of rotatable skirt 26 can be seen extending radially inward towards the central vertical axis C-C (not shown) of the portable base and is latched between the ledge 62 in the rim portion 54 and the shoulder 58. FIG. 2B also illustrates the provision of the sealing 72 on the rim portion 54. It is to be understood that when the lid unit is placed on top of the canister unit, the neck portion 38 of the canister unit 22 enters the space between the sealing 72 on the rim portion and the rotatable skirt 26. When the rotatable skirt 26 is then subsequently moved/rotated, the rotational movement makes the complementary threads of the canister unit and the lid unit engage and thereby forces the lid unit 24 closer to the shoulder portion 46 of the canister unit 22, whereby the sealing 72 is moved in axial direction (downward when closing the portable base) along and into sealing engagement with the radially inward facing surface of the neck portion 38 of the canister unit 22, such as seen at position 73 (see FIG. 3 and in particular FIG. 3B).

FIG. 3 is a cross-sectional view of one embodiment of the portable base 22 in a sealed/closed configuration, i.e., wherein the lid unit 24 is in engagement with the canister unit 22. In FIG. 3 (see also enlarged views of FIGS. 3B & 3C), the rotatable skirt 26 includes a second flange 68. In the illustrated embodiments, the second flange 68 provides a second thread or thread segment of the rotatable skirt 26. In FIG. 3, the first protrusion or thread 48 of the neck portion 38 of the canister unit 22 and the second thread 68 of the rotatable skirt 26 are configured as complementary threads. As such, they are adapted to engage with each other to make the lid unit 24 and the canister unit 22 securely grip to each other when the user is shifting the portable base to the closed/sealed configuration.

In the illustrated views of FIGS. 3, 3B and 3C, the second flange or thread 68 of the rotatable skirt 26 extends in a direction parallel to a plane which is generally perpendicular to the central vertical axis C-C. The second flange 68 extends radially inward towards the central vertical axis and is configured to engage with the canister unit 22 during the closing (sealing) and opening (unsealing) actions of the portable base 20. In the embodiment of FIG. 3B, the second flange 68 slides in the neck portion 38 of the canister unit 22. In one embodiment as illustrated in FIG. 3B, the second flange 68 of the rotatable skirt 26 engages with and slides in a groove or track 70 of the canister unit 22. In the embodiment of FIG. 3B, the groove or track 70 is provided in the neck portion 38 of the canister unit. In the embodiment of FIG. 3B, the groove or track 70 is formed by and/or between the neck portion 38 and the shoulder portion 46 of the canister unit 22. The groove or track 70 is further delimited by the protrusion or first thread 48 of neck portion 38 of the canister unit 22 and the shoulder portion 46 of the canister unit. In the embodiment illustrated in FIG. 3B, the second flange or thread 68 of the rotatable skirt 26 has a shorter radial extent than the first flange or thread 60 of the rotatable skirt 26. The engagement between the first flange 60 of the rotatable skirt 26, the ledge 62 in the rim portion 54 and the shoulder 58 provides for the rotatable skirt 26 to be able to rotate at least partially around the central vertical axis C-C of the portable base 22, and further helps prevent the rotatable skirt 26 from disengaging from the lid unit 24. In the illustrated embodiment, the groove or track 70 of the neck portion 38 and the ledge 62/shoulder 58 of the rim portion 54 can be understood to provide “female” members, and the first thread 60 and the second thread 68 of the rotatable skirt 26 to provide “male” members, of the complementary threads.

In embodiments, and with part reference to FIG. 4, the protrusion or first thread 48 of the neck portion 38 of the canister unit 22 includes four evenly distributed, individual, and finite thread segments, of which three 48a, 48b, 48c are visible in FIG. 4. The segment(s) 48a, 48b, 48c of the first thread are sloped or tapered such that by the rotational movement of the skirt 26, comprising the second thread(s) 68 (FIG. 3), in relation to the complementary first, sloping thread segment(s) 48a, 48b, 48c, the lid unit 24 is moved in an axial direction parallel to the central vertical axis (i.e., when carrying out the opening or closing action). In the illustrated embodiments, the segments 48a, 48b, 48c of the first thread on the neck portion 38 are provided on a radially outward facing surface 39 of the neck portion.

Also illustrated in FIGS. 1, 2, 2B, 3, 3B and 3C, is a sealing 72 provided at the rim portion 54 of the lid unit 24 and adapted to seal between the canister unit 22 and the lid unit 24. The sealing 72 is attached to the rim portion 54 (see e.g., FIG. 2B) and adapted to be brought into engagement with a radially inward facing surface of the neck portion 38 of the canister unit 22 when the lid unit 24 is placed on top thereof (e.g., FIGS. 3B, 3C). When the sealing 72 is moved axially downward by rotation of the rotatable skirt 26 and the complementary first and second threads 48, 68 are engaging with each other, the sealing is urged or pressed into engagement with the radially inward facing surface of the neck portion 38 to create a sealing effect, whereby the portable base 22 is shifted to the sealed configuration. The rotation of the skirt 26 and the resulting downward, vertical movement of the lid unit 24 leads to increased pressure between the material of the sealing 72 and the radially inward facing surface of the neck portion 38 which is sufficient to create an air- and liquid-tight seal between the lid unit 24 and the canister unit 22.

In the embodiment of FIG. 3B particularly, the sealing 72 is provided at a location of the rim portion 54 being closer to the bottom 24b of the lid unit 24 than to the top 24a of the lid unit (taken along the central vertical axis). The sealing 72 seals against (at position 73) a radially inward facing surface of vertical flange 42 of the neck portion 38 of the canister unit 22 in the sealed configuration of the portable base 20. The radially inward facing surface of the vertical flange 42 is generally parallel to the central vertical axis C-C and has an extent in the vertical direction parallel to the central vertical axis, thus providing an abutment surface for the sealing 72 against the neck portion 38 of the canister unit 22 at position 73. In FIG. 3B, a tapering 44 of the vertical flange 42 of the neck portion 38 is shown to be formed at an uppermost end portion of the vertical flange 42 of the neck portion 38 and forms an angularly offset portion relative to the central vertical axis C-C. In the embodiments of the illustrated views, the sealing 72 is provided as an O-ring (annular sealing ring) and extends annularly around the rim portion 54 of the lid unit 24.

In the embodiments of FIGS. 1, 3 and 3C, the rotatable skirt 26 is shown to include a gripping tab 27 extending in a direction radially outward in relation to the central vertical axis C-C. The gripping tab 27 extends radially beyond the lateral wall 28 forming an annular sidewall of the lid unit 24. The gripping tab 27 extends radially beyond the shoulder portion 46 of the canister unit 22. Thereby, access to the gripping tab 27 is easy and unobstructed for the user. The opening or closing actions of the portable base by rotating the skirt 26 can conveniently be carried out by pushing the gripping tab 27 in one direction with the fingers of one hand and holding on to the finger-hold 23 of the canister unit 22 with the fingers of the other hand.

In the embodiments illustrated in FIGS. 2 and 3, the lid unit 24 includes a through-going passage 76 for filling irrigation liquid into the volume 78 in the canister unit 22. The through-going passage 76 is provided generally parallel to the central vertical axis C-C. Further, in embodiments and as further illustrated in FIGS. 1 and 4, access to the irrigation liquid passage 76 can be provided via cap 32. The cap 32 is provided in the top 24a of the lid unit 24. As shown in FIGS. 2 and 3, the cap 32 can include an internal sealing 80 for assisting in providing the air- and liquid-tight seal between the lid unit 24 and the canister unit 22 during use of the portable base 22. The cap 32 facilitates filling (or emptying) of the canister unit 22 with irrigation liquid without requiring removal of the lid unit 24 entirely from the canister unit 22. In embodiments as illustrated by FIGS. 2 and 3, the lid unit 24 is shown to include at least one internal chamber 84 adapted to contain one or more control elements (not shown) of a system for irrigating the bowels of a user. Also, on the top 24a of the lid unit 24 a connection 86 for engagement with tubing of a system for irrigation of the bowels of a user is provided. The connection 86 also forms a passage for allowing the irrigation liquid from the canister unit 22 to exit by means of a pumping action provided by a pump of the system for irrigating the bowels of a user (which pump could conveniently be provided in the internal chamber 84).

FIG. 5 is a schematic, perspective view of a simplified model of one embodiment of a portable base 22 and is primarily intended to illustrate the rotational opening/closing principle of the portable base. The portable base 22 can for example be made from a polymeric material suitable for the operating temperatures of the irrigation liquid and/or for temperatures high enough to kill microorganisms. FIG. 5 shows the lid unit 24 with the rotatable skirt 26 and the gripping tab 27 of the skirt 26. The lid unit 24 is shown in a situation where it is separated from the canister unit 24. Rotation arrow A indicates one example of how the rotatable skirt 26 can be rotated for closing/sealing and (vice versa in the opposite direction of arrow A) for opening/unsealing actions by pushing on the gripping tab 27 and rotating the skirt 26 in relation to the canister unit 22, such that the complementary second threads of the skirt 26 (not visible in FIG. 5), and the first threads or thread segments 48a-d of the canister unit 22, can be brought into engagement with each other. As the model in FIG. 5 is simplified, cap 32 and the connection for engagement of tubing 86, are not shown. In embodiments the canister unit 22 in FIG. 5 can also include a tab or finger-hold 23 provided on the sidewall of the canister unit to further assist the user in opening and closing the portable base 20 as described above.

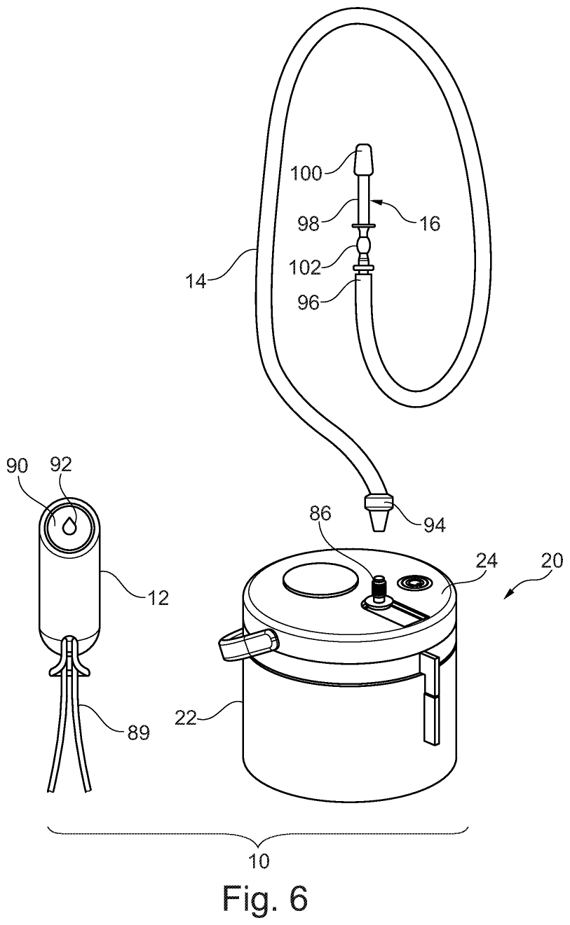

FIG. 6 is a schematic, perspective view illustrating components of one embodiment of a system 10 for irrigating the bowels of a user. In FIG. 6, the system 10 is shown in a not fully assembled condition.

In FIG. 6, the system 10 includes a portable base 20 according to embodiments of the first aspect of the disclosure. The system 10 can include a remote-control unit 12 adapted to communicate wirelessly with a control component (not shown) of the portable base 20 to control the irrigation system. In embodiments, either the remote-control unit 12 or the control component or both, includes a central processing unit (CPU). The remote-control unit 12 includes a cord or string 89 for attachment around the wrist of the user or similar. In the embodiment of FIG. 6, the remote-control unit 12 includes one button 90. The remote-control unit 12 and/or the button 90 can include indicia or pictograms 92 indicating one or more functions and/or instructions to a user of the system, such as, but not limited to, the possibility of releasing a flow of irrigation liquid from the canister unit 22 for commencement of a bowel irrigation cycle.

FIG. 6 illustrates one embodiment of a system including the canister unit 22 and the lid unit 24 of the portable base 22 disclosed herein, and further tubing 14, and a catheter or anal probe 16. A first end portion 94 of the tubing 14 is configured for connection and/or attachment with the connection 86 on the lid unit 24. A second end portion 96 of the tubing 14 is configured for connection and/or attachment with the anal probe 16. These connections assist in providing fluid communication between the irrigation liquid in the canister unit 22 and anal probe 16 via the tubing 14. The catheter or anal probe 16 is configured to be inserted into the rectum of a user. The anal probe 16 includes a catheter portion 98 for insertion into the rectum of the user to release irrigation liquid via the catheter into the bowels of the user. The catheter portion 98 includes a catheter tip 100. The anal probe 16 further includes a handle portion 102, which is connectable to the tubing 14. Although not illustrated in FIG. 6, the system 10 can optionally include a set of instructions for use, and/or a container for storing the components of the system.

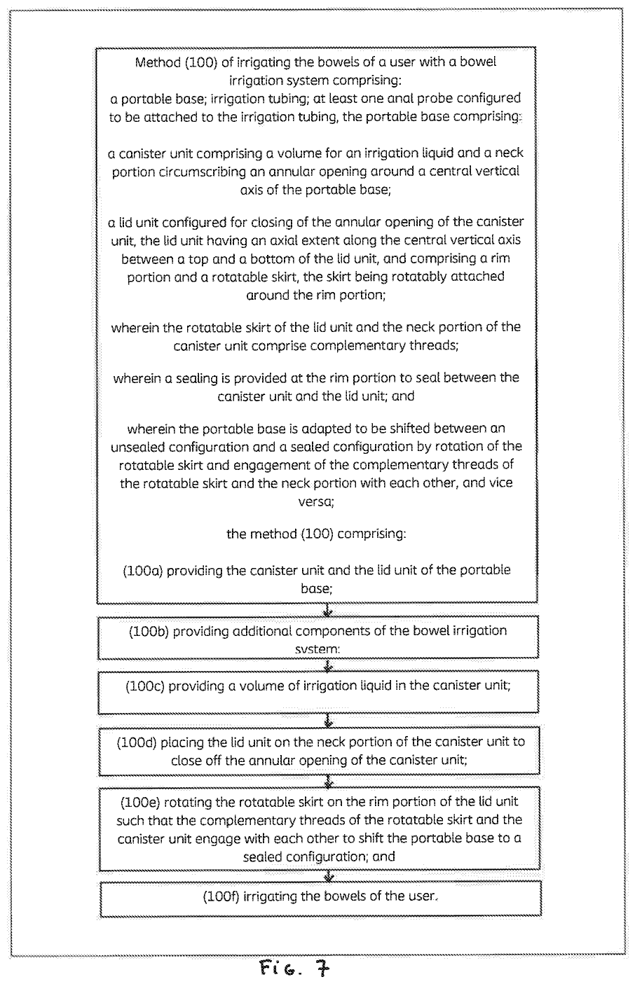

FIG. 7 is a diagram illustrating the steps (100a)-(100f) of a method (100) of irrigating the bowels of a user with a bowel irrigation system incorporating a portable base according to the first aspect.

Although particular features have been shown and described, it will be understood that they are not intended to limit the claimed invention, and it will be made obvious to those skilled in the art that various changes and modifications may be made without departing from the scope of the claimed invention. The specification and drawings are, accordingly, to be regarded in an illustrative rather than restrictive sense. The claimed invention is intended to cover all alternatives, modifications, and equivalents.

### INDEX OF REFERENCE NUMBERS

10 system for irrigating the bowels of a user
12 remote-control unit
14 tubing
16 anal probe or catheter
20 portable base
22 canister unit
23 tab or finger-hold (of canister unit)
24 lid unit
24a top of the lid unit
24b bottom of the lid unit
25 bottom portion of canister unit
26 rotatable skirt
27 gripping tab (of rotatable skirt)
28 lateral wall
30 sidewall of canister unit
32 door (of lid unit)
34 handle
36 hinge
38 neck portion (of canister unit)
39 outward facing surface (of neck portion 38)
40 annular opening
42 vertical flange (of neck portion)
44 tapering of vertical flange
46 shoulder portion (of canister unit)
48 protrusion/first thread (of neck portion)
50 non-planar underside (of canister unit)
52 apex
54 rim portion (of lid unit)
55 upper portion (of lateral wall 28)
58 shoulder (of rim portion)
60 first flange (of rotatable skirt)
62 ledge (of rim portion)
64 projection
66 outer periphery of lateral wall
68 second flange/second thread
70 groove or track (in neck portion)
72 sealing
73 position of seal
76 through-going passage
78 volume of the canister unit
80 internal sealing (of door)
82 gripping tab (of door)
84 internal chamber
86 connection for engagement of tubing
88 power-charging interface
89 cord or string for remote-control
90 button
92 indicia
94 first end portion of tubing
96 second end portion of tubing
98 catheter portion
100 catheter tip
102 handle portion

1. A portable base for a system for irrigating the bowels of a user, comprising:
a canister unit comprising a volume for an irrigation liquid and a neck portion circumscribing an annular opening around a central vertical axis of the portable base; and
a lid unit configured for closing of the annular opening of the canister unit, the lid unit having an axial extent along the central vertical axis between a top and a bottom of the lid unit, and comprising a rim portion and a rotatable skirt, the skirt being rotatably attached around the rim portion;
wherein the rotatable skirt of the lid unit and the neck portion of the canister unit comprise complementary threads;
wherein a sealing is provided at the rim portion and configured to seal between a radially inward facing surface of the neck portion of the canister unit and the lid unit;
wherein the portable base is adapted to be shifted between an unsealed configuration and a sealed configuration by rotation of the rotatable skirt and engagement of the complementary threads of the rotatable skirt with the neck portion.

2. The portable base of claim 1, wherein, when the rotatable skirt is engaged with the neck portion, an air and liquid tight seal is formed between the neck portion and the lid unit and the canister unit is pressurizable to a pressure in a range from 1-23 psi.

3. The portable base of claim 1, wherein the annular opening of the canister unit has a diameter of between 100-150 mm.

4. The portable base of claim 1, wherein the sealing is provided at a location of the rim portion closer to the bottom of the lid unit than to the top of the lid unit.

5. The portable base of claim 1, wherein the sealing engages and seals against a lowermost end of the radially inward facing surface of the neck portion in the sealed configuration of the portable base.

6. The portable base of claim 1, wherein the sealing comprises an O-ring.

7. The portable base of claim 1, wherein first threads on the neck portion comprise at least three spaced apart thread portions provided on a radially outward facing surface of the neck portion.

8. The portable base of claim 1, wherein the rotatable skirt comprises at least one second thread portion provided on a radially inward facing surface of the skirt.

9. The portable base of claim 1, wherein the rotatable skirt includes a first flange configured to engage with a ledge of the rim portion of the lid unit to allow the first flange of the skirt to slide on the ledge during rotation of the skirt.

10. The portable base of claim 9, wherein the rotatable skirt further comprises a gripping tab extending in a direction radially outward in relation to the central vertical axis.

11. The portable base of claim 1, wherein the lid unit comprises at least one internal chamber.

12. The portable base of claim 11, wherein the at least one internal chamber comprises one or more control elements of a system for irrigating the bowels of a user.

13. A bowel irrigation system comprising the portable base of claim 1 and further comprising: irrigation tubing comprising a first end and a second end, the first end of the irrigation tubing configured to be connected to the lid unit of the portable base and an probe configured to be connected to the second end of the irrigation tubing.

14. Method of irrigating the bowels of a user with a bowel irrigation system of claim 13, comprising the steps of:
(a) providing the canister unit and the lid unit of the portable base;
(b) providing additional components of a bowel irrigation system;
(c) providing a volume of irrigation liquid in the canister unit;
(d) placing the lid unit on the neck portion of the canister unit to close the annular opening of the canister unit;
(e) rotating the rotatable skirt on the rim portion of the lid unit such that the complementary threads of the rotatable skirt and the canister unit engage with each other to shift the portable base to a sealed configuration; and
(f) irrigating the bowels of the user.

15. Method of sealing a canister unit of a bowel irrigation system of claim 13, comprising the steps of:
(a) providing the canister unit and the lid unit of the portable base;
(b) providing additional components of a bowel irrigation system;
(c) providing a volume of irrigation liquid in the canister unit;
(d) placing the lid unit on the neck portion of the canister unit to close the annular opening of the canister unit; AND
(e) rotating the rotatable skirt on the rim portion of the lid unit such that the complementary threads of the rotatable skirt and the canister unit engage with each other to shift the portable base to a sealed configuration.

16. The portable base of claim 1, wherein the rim portion is closer to the central vertical axis than the rotatable skirt is to the central vertical axis, and the rotatable skirt rotates and moves independent from movement of the rim portion and independent from the canister unit.

17. The portable base of claim 1, further comprising:
a cap formed in the top of the lid;
wherein the cap is removable from the lid to allow addition of the irrigation liquid into the volume of the canister unit while leaving the lid unit is closed relative to the canister unit.

18. The portable base of claim 1, wherein the canister unit comprises a sidewall that extends from the bottom of the canister unit to a shoulder of the canister unit, and the shoulder of the canister unit positions the neck portion of the canister between the rotatable skirt of the lid unit and the rim portion of the lid unit;
wherein the rotatable skirt of the lid unit is located outside the rim portion of the lid unit with the rim portion of the lid unit positioned closer to the central vertical axis than the rotatable skirt is to the central vertical axis; and
wherein rotation of the rotatable skirt forces the lid unit in a downward direction closer to the shoulder of the canister unit.

19. The portable base of claim 18, wherein the sealing comprises an annular seal disposed around the rim portion of the lid unit to position the annular seal between the rim portion of the lid unit and the neck portion of the canister;
wherein the rotation of the rotatable skirt forces the annular seal to move in an axial direction downward and into a sealing engagement with a radially inward facing surface of the neck portion of the canister.
